(19) United States
(12) Patent Application Publication
(10) Pub. No.: US 2010/0179428 A1
(43) Pub. Date: Jul. 15, 2010

(54) VIRTUAL INTERACTIVE SYSTEM FOR ULTRASOUND TRAINING

(75) Inventors: Peder C. Pedersen, Sterling, MA (US); Thomas L. Szabo, Newburyport, MA (US); Christian J. Banker, Encinitas, CA (US)

Correspondence Address: BURNS & LEVINSON, LLP, 125 SUMMER STREET, BOSTON, MA, 02110, US

(73) Assignees: Worcester Polytechnic Institute, Worcester, MA (US); The Trustees of Boston University, Boston, MA (US)

(21) Appl. No.: 12/728,478

(22) Filed: Mar. 22, 2010

Related U.S. Application Data
(63) Continuation-in-part of application No. PCT/US09/37406, filed on Mar. 17, 2009.
(60) Provisional application No. 61/037,014, filed on Mar. 17, 2008.

(51) Int. Cl. A61B 8/14; G09B 23/28

(52) U.S. Cl. 600/443; 434/262

(57) ABSTRACT

A virtual interactive ultrasound training system for training medical personnel in the practical skills of performing ultrasound scans, including recognizing specific anatomies and pathologies.

### CROSS REFERENCE TO RELATED APPLICATIONS

This application is a continuation-in-part of PCT Patent Application Serial Number PCT/U.S.09/37406, entitled VIRTUAL INTERACTIVE SYSTEM FOR ULTRASOUND TRAINING, filed on Mar. 17, 2009, which claims the priority date of Provisional Application Ser. No. 61/037,014, entitled VIRTUAL INTERACTIVE SYSTEM FOR ULTRASOUND TRAINING, filed on Mar. 17, 2008, both of which this application incorporates by reference in their entirety.

### BACKGROUND

Simulation-based training is a well-recognized component in maintaining and improving skills. Consequently, simulation-based training is critically important for a number of professionals, such as airline pilots, fighter pilots, nurses and medical surgeons, among others. Such skills require hand-eye coordination, spatial awareness, and integration of multi-sensory input, such as tactile and visual. People in these professions have been shown to increase their skills significantly after undergoing simulation training.

A number of medical simulation products for training purposes are on the market. They include manikins for CPR training, obstetrics manikins, and manikins where chest tube insertion can be practiced, among others. There are manikins with an arterial pulse for assessment of circulatory problems or with varying pupil size for practicing endotracheal intubation. In addition, there are medical training systems for laparoscopic surgery practice, for surgical planning (based on three-dimensional imaging of the existing condition), and for practicing the acquisition of biopsy samples, to name just a few applications.

Ultrasound imaging is the only interactive, real time imaging modality. Much greater skill and experience is required for a sonographer to acquire and store ultrasound images for later analysis than for performing CT or MRI scanning Effective ultrasound scanning and diagnosis based on ultrasound imaging requires anatomical understanding, knowledge of the appearance of pathologies and trauma, proper image interpretation relative to transducer position and orientation on the patient's body, the effect of compression on the patient's body by a transducer, and the context of the patient's symptoms.

Such skills are today primarily obtained through hands-on training in medical school, at sonographer training programs, and at short courses. These training sessions are an expensive proposition because a number of live, healthy models, ultrasound imaging systems, and qualified trainers are needed, which detract from their normal diagnostic and revenue-generating activities. There are also not enough teachers to meet the demand because qualified sonographers and physicians are required to earn Continuing Medical Examination (“CME”) credits annually.

Various ultrasound phantoms have been developed and are widely used for medical training purposes, such as prostate phantoms, breast phantoms, fetal phantoms, phantoms for practicing placing IV lines, etc. There are major limitations to the use of these phantoms for ultrasound training purposes. First, they need to be used together with an available ultrasound scanner. Thus, such simulation training can only occur at the hospital and only when the ultrasound scanner is not otherwise used for patent examination. Second, with a few exceptions, phantoms are not generally available for training to recognize trauma and pathology situations. Thus, formal automated training to locate an inflamed pancreas, find gallstones, determine abnormal fetal development, detect venous thrombosis, to name a few, is generally not available. When a trauma case occurs, treatment is of course paramount, and there is no time available for training. In addition, these phantoms are static or have specialized parts, and so fall short of simulating a dynamic, interactive human.

Given the ubiquitous use of ultrasound for medical diagnosis and the large number of potential users, there is a large and unmet need for cost-effective ultrasound training Training needs comes in several forms, including: (i) training active users in using new ultrasound scanners; (ii) training active users in new diagnostic procedures; (iii) training active users for re-certification, to maintain skills and earn continuing medical education credit on an annual basis; and (iv) training new users, such as primary care physicians, emergency medicine personnel, paramedics and EMTs.

What is needed is a better system and method of use that can help train ultrasound operators on a wide-range of diagnostic subjects in a cost-effective, realistic, and consistent way.

### SUMMARY

The needs set forth herein as well as further and other needs and advantages are addressed by the present embodiments, which illustrate solutions and advantages described below.

The method of present embodiment for generating ultrasound training image material can include, but is not limited to including, the steps of scanning a living body with an ultrasound transducer to acquire more than one at least partially overlapping ultrasound 3D image volumes/scans, tracking the position/orientation of the ultrasound transducer while the ultrasound transducer scans in a preselected number of degrees of freedom, storing the more than one at least partially overlapping ultrasound 3D image volumes/scan and the position/orientation on computer readable media, and stitching the more than one at least partially overlapping ultrasound 3D image volumes/scans into one or more 3D image volumes based on the position/orientation.

The tracking may take place over the body surface of a physical manikin, or it may take place over a scanning surface, emulating a specific region of a virtual subject appearing on the same screen as the ultrasound image or on a different screen from the ultrasound image. In the case of tracking the position and orientation of the mock transducer over a scanning surface, a virtual transducer on the surface of a virtual subject is moved correspondingly.

The method can optionally include the steps of inserting and stitching at least one other ultrasound scan into the one or more 3D image volumes, storing a sequence of moving images (4D) as a sequence of the one or more 3D image volumes each tagged with time data, digitizing data corresponding to an manikin surface of the manikin, recording the digitized surface on a computer readable medium represented as a continuous surface, and scaling the one or more 3D image volumes to the size and shape of the manikin surface of the manikin.

Optionally, the virtual subject can have the exact body appearance of the human subject, who was scanned to produce the image data. This can be accomplished by moving a tracking system that is attached to the transducer in a relatively closely-spaced grid pattern over the body surface tracking data, possibly not collecting image data. These tracking data can be captured by, for example, ts_capture software, and can be provided to a conventional computer system, such as, for example, a user-contributed library, gridfit, from MATLAB®'s File Exchange, that can reconstruct the body surface based on the tracking data. Ultimately, a user can choose an image from a library of, for example, pathological condition images, and associated with the selected image is body surface information of a selected type, for example, a sixty year old male having a kidney abnormality. As a result of the present teachings, an exact body size can accompany the image volume of a given pathological condition, when the virtual subject is used for training instead of the manikin.

The image acquisition system of the present embodiment can include, but is not limited to including an ultrasound transducer and associated ultrasound imaging system, at least one 6 degrees of freedom tracking sensor integrated with the ultrasound transducer/sensor, a volume capture processor generating a position/orientation of each image frame contained in the ultrasound scan relative to a reference point, and producing at least one 3-D volume obtained with the ultrasound scan, and a volume stitching processor combining a plurality of the at least two 3-D volumes into one composite 3D volume. The system can optionally include a calibration processor establishing a relationship between output of the ultrasound transducer/sensor and the ultrasound scan and a digitized surface of a manikin, an image correction processor applying image correction to the ultrasound scan when there is tissue motion, resulting in the 3D volume reflecting tissue motion correction, and a numerical model processor acquiring a numerical virtual model of the digitized surface, and interpolating and recording the digitized surface, represented as a continuous surface, on a computer readable medium.

The ultrasound training system of the present embodiment can include, but is not limited to including, one or more scaled 3-D image volumes stored on electronic media, the image volumes containing 3D ultrasound scans recorded from a living body, a manikin, a 3-D image volume scaled to match the size and shape of the manikin, a mock transducer having sensors for tracking a mock position/orientation of the mock transducer relative to the manikin in a preselected number of degrees of freedom, an acquisition/training processor having computer code calculating a 2-D ultrasound image from the based on the position/orientation of the mock transducer, and a display presenting the 2-D ultrasound image for training an operator.

Alternatively, the ultrasound training system of the present embodiment can include a virtual subject in the place of a manikin, this virtual subject being displayed in 3D rendering on a computer screen. When the body appearance of the virtual subject is an exact replica of the human being that was scanned for the ultrasound image volume, no scaling is need to scale the image volume to fit the virtual subject. The virtual subject can be scanned by a virtual transducer, whose position and orientation appears on the body surface of the virtual subject and whose position and orientation are controlled by the trainee by moving a sham transducer over a scan surface. This scan surface can have the mechanical compliance approximating that of a soft tissue surface, for example, a skin-like material backed by ½ inch to 1 inch of appropriately compliant foam material. If optical tracking is used, then the skin surface must have the necessary optical tracking characteristics. Alternatively, a graphic tablet such as, for example, but not limited to, the WACOM® tablet can be used, covered with the compliant foam material and a skin-like surface. As a further alternative, the scanning surface can be embedded with a dot pattern, such as, for example, the ANOTO® dot pattern, as used with a digital paper and digital pen.

The acquisition/training processor can record a training scan pattern and a sequence of time stamps associated with the position and orientation of the mock transducer, scanned by the trainee on the manikin or on the scan pad surface, compare a benchmark scan pattern, scanned by an experienced sonographer, of the manikin with the training scan pattern, and store results of the comparison on the electronic media. The system can optionally include a co-registration processor co-registering the 3-D image volume with the surface of the manikin in 6 DOF by placing the mock transducer at a specific calibration point or placing a transmitter inside the manikin, a pressure processor receiving information from pressure sensors in the mock transducer, and a scaling processor scaling and conforming a numerical virtual model to the actual physical size of the manikin as determined by the digitized surface, and modifying a graphic image based on the information when a force is applied to the mock transducer and the manikin surface of the manikin. The system can further optionally include instrumentation in or connected to the manikin to produce artificial physiological life signs, wherein the display is synchronized to the artificial life signs, changes in the artificial life signs, and changes resulting from interventional training exercises, a position/orientation processor calculating the 6 DoF position/orientation of the mock transducer in real-time from a priori knowledge of the manikin surface and less than 6 DoF position/orientation of the mock transducer on the manikin surface, an interventional device fitted with a 6 DoF tracking device that sends real-time position/orientation to the acquisition/training processor, a pump introducing artificial respiration to the manikin, the pump providing respiration data to an mock transducer processor, a image slicing/rescaling processor dynamically rescaling the 3-D ultrasound image to the size and shape of the manikin as the manikin is inflated and deflated, and an animation processor representing an animation of the interventional device inserted in real-time into the 3-D ultrasound image volume.

The method of the present embodiment for evaluating an ultrasound operator can include, but is not limited to including, the steps of storing a 3-D ultrasound image volume containing an abnormality on electronic media, associating the 3-D ultrasound image volume with a manikin or a virtual subject (together referred to herein as a body representation), receiving an operator scan pattern associated with the body representation from a mock transducer, tracking mock position/orientation of the mock transducer in a preselected number of degrees of freedom, recording the operator scan pattern using the mock position/orientation, displaying a 2-D ultrasound image slice from the 3-D ultrasound image volume based upon the mock position/orientation, receiving an identification of a region of interest associated with the body representation, assessing if the identification is correct, recording an amount of time for the identification, assessing the operator scan pattern by comparing the operator scan pattern with an expert scan pattern, and providing interactive means for facilitating ultrasound scanning training. The method can optionally include the steps of downloading lessons in image-compressed format and the 3-D ultrasound image volume in image compressed format through a network from a central library, storing the lessons and the 3D ultrasound image volume on a computer-readable medium, modifying a display of the 3-D ultrasound image volume corresponding to interactive controls in a simulated ultrasound imaging system control panel or console with controls, displaying the location of an image plane in the 3-D ultrasound image volume on a navigational display, and displaying the scan path based on the digitized representation of the body representation surface of the body representation.

Other embodiments of the system and method are described in detail below and are also part of the present teachings.

For a better understanding of the present embodiments, together with other and further aspects thereof, reference is made to the accompanying drawings and detailed description, and its scope will be pointed out in the appended claims

### BRIEF DESCRIPTION OF THE DRAWINGS

FIG. 1 is a pictorial depicting one embodiment of the method of generating ultrasound training material;

FIG. 2A is a pictorial depicting one embodiment of the ultrasound training system;

FIG. 2B is a pictorial depicting the conceptual appearance of interactive training system with virtual subject;

FIG. 2C is a block diagram depicting the main components of interactive training system with virtual subject;

FIG. 2D is a pictorial depicting the compliant scan pad with built-in position sensing; mock transducer with Micro-Electro-Mechanical Systems (MEMS)-based angle sensing capabilities;

FIG. 2E is a pictorial depicting the compliant scan pad without built-in position sensing mock transducer with optical position sensing and MEMS-based angle sensing capabilities;

FIG. 3 is a block diagram describing another embodiment of the ultrasound training system;

FIG. 4 is a block diagram describing yet another embodiment of the ultrasound training system;

FIG. 5 is a pictorial depicting one embodiment of the graphical user interface for the display of the ultrasound training system;

FIG. 6 is a block diagram describing one embodiment of the method of distributing ultrasound training material;

FIG. 7 is a pictorial depicting one embodiment of the manikin used with the ultrasound training system;

FIG. 8 is a block diagram describing one embodiment of the method of stitching an ultrasound scan;

FIG. 9 is a block diagram describing one embodiment of the method of generating ultrasound training image material;

FIG. 10 is block diagram describing one embodiment of the mock transducer pressure sensor system;

FIG. 11 is a block diagram describing one embodiment of the method of evaluating an ultrasound operator;

FIG. 12 is a block diagram describing one embodiment of the method of distributing ultrasound training material; and

FIG. 13 is a block diagram of another embodiment of the ultrasound training system.

### DETAILED DESCRIPTION

The present teachings are described more fully hereinafter with reference to the accompanying drawings, in which the present embodiments are shown. The following description is presented for illustrative purposes only and the present teachings should not be limited to these embodiments.

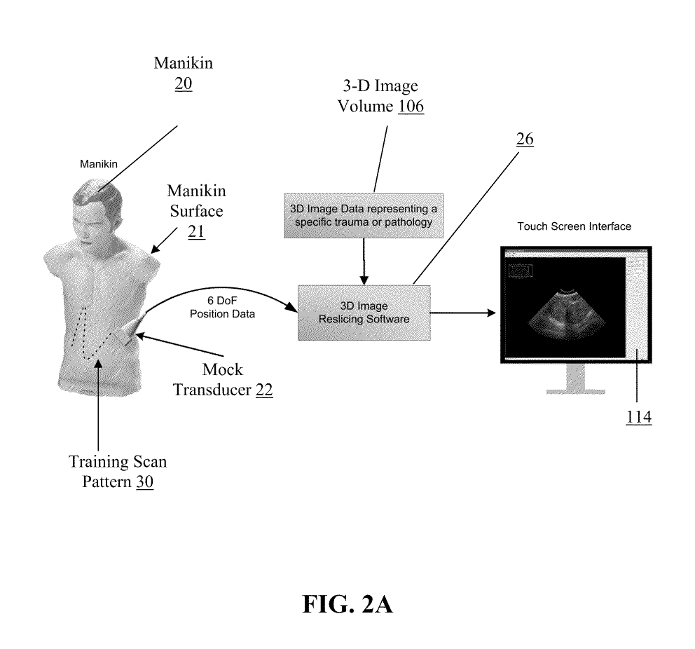

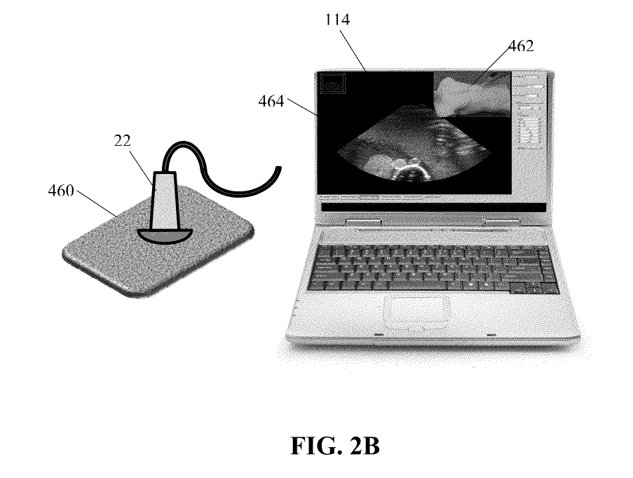

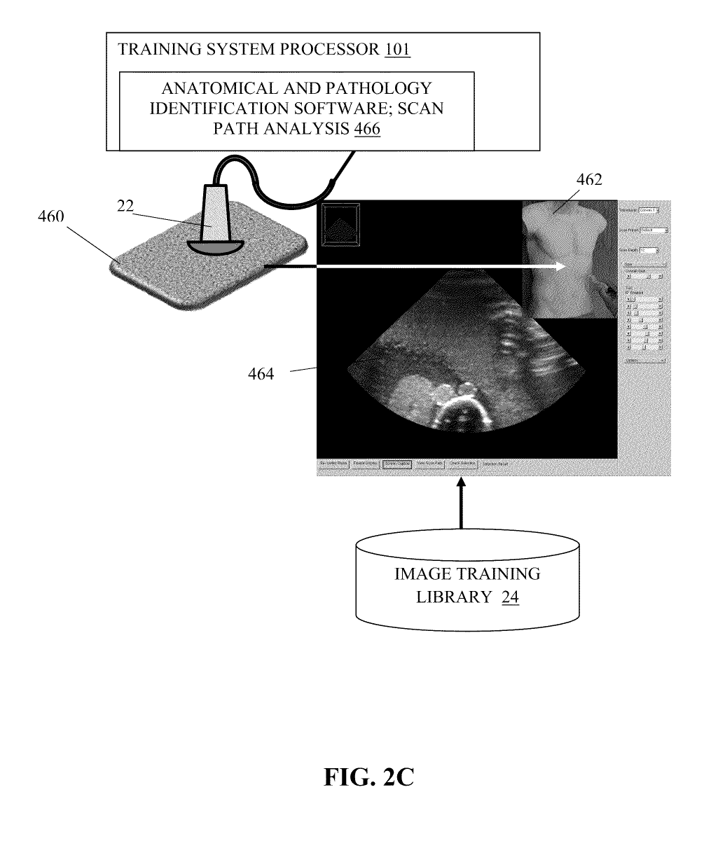

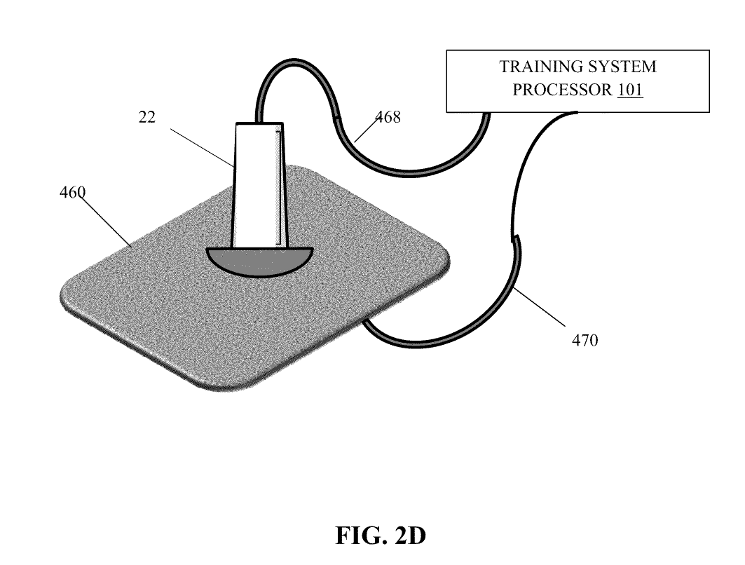

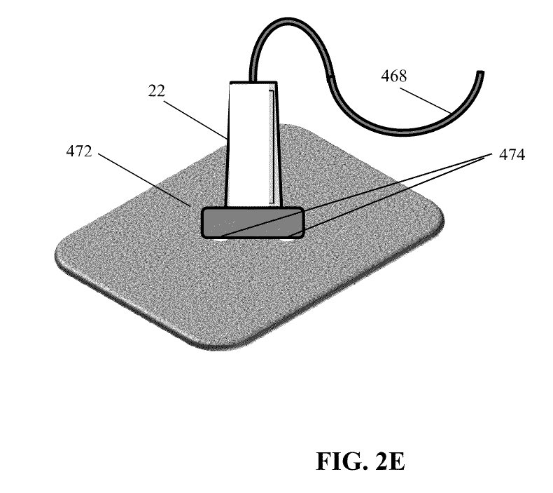

Previous ultrasound simulators are expensive, dedicated systems that present barriers to widespread use. The system described herein is a simple, inexpensive approach that enables simulation and training in the convenience of an office home or training environment. The system may be PC-based and computers used in the office or at home for other purposes can be used for the simulation of ultrasound imaging as described below. In addition, an inexpensive manikin representing a body part such as a torso (possibly with a built-in transmitter), a mock ultrasound transducer with tracking sensors, and the software described below help complete the system (shown in FIG. 2A).

An alternative embodiment can be achieved by scanning with a mock transducer over scan surface with the mechanical characteristics of a soft tissue surface. The mock transducer alone may implement the necessary 5 DoF, or the 5 DoF may be achieved through linear tracking integrated in the scan surface or linear tracking by optical means on the scan surface and angular tracking integrated into the mock transducer. The movements of the mock transducer over the scan surface are visualized in the form of a virtual transducer moving over the body surface of a virtual subject.

The simplicity of this approach makes it possible to create low-cost simulation systems in large numbers. In addition, the 3-D ultrasound image volumes used for the training system can be easily mass reproduced and made downloadable over the Internet as described below.

When using a physical manikin, the sensors of the tracking systems described herein are referred to as external sensors because they require external transmitters in addition to tracking sensors integrated into the mock transducer handle. In contrast, self-contained tracking sensors can be used either with the physical manikin or with scan surface (scan pad) in combination with the virtual subject and the virtual transducer. These sensors only require that sensors be integrated into a mock transducer handle in order to determine the position and the orientation of the transducer with five degrees of freedom, although not limited thereto. The self-contained tracking sensors can be connected either wirelessly or by standard interfaces such as USB to a personal computer. Thus, the need for external tracking infrastructure is eliminated. Alternatively, external tracking can be achieved through image processing, specifically by measuring the degree of image decorrelation. However, such decorrelation may have a variable accuracy and may not be able to differentiate between the transducer being moved with a fixed orientation or being angled at a fixed position.

The sensors in the self-contained tracking system may be of a MEMS-type and an optical type, although not limited thereto. An exemplary tracking concept is described in International Publication No. WO/2006/127142, entitled Free-Hand Three-Dimensional Ultrasound Diagnostic Imaging with Position and Angle Determination Sensors, dated Nov. 30, 2006 (142), which is incorporated by reference herein in its entirety. The position of the mock transducer on the surface of a body representation may be determined through optical sensing, in a principle similar to an optical mouse that uses the cross-correlation between consecutive images captured with a low-resolution CCD array to determinate change in position. However, for the sake of a compact design near the phantom surface, the image may be coupled from the surface to the CCD array via an optical fiber bundle. Excellent tracking has been demonstrated. Very compact, low-power angular rate sensors are now available to determine the orientation of the transducer along three orthogonal axes. Occasionally, however, the transducer may need to be placed in a calibration position to minimize the influence of drift.

The optical tracking described above is a single optical tracker, which can provide position information, but has no redundancy. In contrast, a dual optical tracker, which can include, but is not limited to including, two optical tracking computer mice, one in each end of the mock transducer, provides two advantages: if one optical tracker should loose position tracking because one end of the sham transducer is momentarily lifted, the other can maintain tracking. In addition, a dual optical tracker can determine rotation and can provide redundancy for the MEMS rotation sensing. For example, using an optical mouse, an image of the scanned surfaced can be captured as is known in the art. If two computer mice are attached, a dual optical tracker device can be constructed which can detect rotation (see '142). A third alternative is to embed the scanning surface with a dot pattern, such as, for example, the ANOTO® dot pattern, as used with a digital paper and digital pen as described in U.S. Pat. No. 5,477,012. The dot pattern is non-repeating, and can be read by a camera which can, because of the dot pattern, unambiguously determine the location on the scan surface.

The manikin may represent a certain part of the human anatomy. There may be a neck phantom or a leg phantom for training on vascular imaging, an abdominal phantom for internal medicine, and an obstetrics phantom, among others. In addition, a phantom with cardiac and respiratory movement may be used. This may require a sequence of ultrasound image volumes to be acquired, where each image volume corresponds to a point in time in the cardiac cycle. In this case, due to the data size, the information may need to be stored on a CD-ROM or other storage device rather than downloaded over a network as described below. The manikin can be solid, hollow, even inflatable, as long as it produces an anatomically realistic shape, and it provides a good surface for scanning Optionally, the outer surface may have the touch and feel of a real skin. Another variation of the phantom could be made of transparent “skin” and actually contain organs. Even in this case, there will be no actual scanning, and the location of the organ must correspond to what is seen on the ultrasound training image.

In another embodiment the manikin may not necessarily have the outer shape of a body part but may be a more arbitrary shape such as a block of tissue-mimicking material. This phantom can be used for needle-guidance training. In this case, both the needle and the mock transducer may have five or six DOF sensors and the position of the needle is overlaid on the image plane selected by the orientation and position of the mock transducer. An image of the part of the needle in the image plane may be superimposed on the usual selected cut plane determined by transducer position, described further below. The 3-D image training material can contain a predetermined body of interest, such as an organ or a vessel such as vein, although not limited thereto. Even though the needle goes in the manikin (e.g., smaller carotid phantom) described above, it may not be imaged. Instead, a realistic simulation needle, based on the 3-D position of the needle, can be animated and overlaid on the image of the cut plane.

In a different embodiment, there is no physical manikin, but a virtual subject which exists only in electronic form. Of significance is the fact that the virtual subject will have the exact appearance of the human subject that was scanned to provide the image material. Image material from male and female, young and old, heavy and thin, can be represented by the corresponding body appearance. This exact appearance is acquired through scanning the body surface with the tracking sensor in a closely spaced grid pattern.

The scan pad, on which the trainee moves the mock transducer, can represent a given surface area of the virtual subject. The location on the body surface of the virtual subject that is represented by the scan pad can be highlighted. This location can be shifted to another part of the body surface by the use of arrow keys on the keyboard, by the use of a computer mouse, by use of a finger with a touch screen, by use of voice commands, or by other interactive techniques. Likewise, the area of the body surface represented by the scan pad can correspond to the same area of the body surface of the virtual subject, or to a scaled up or scaled down area of the body surface.

The scan pad may be a planar surface of unchangeable shape, or it may be a curved surface of unchangeable shape, or it may be changeable in shape so it can be modified from a planar surface to a curved surface of arbitrary shape and back to a planar surface.

Finally, the ultrasound training system can be used with an existing patient simulator or instrumented manikin. For example it can be added to a universal patient simulator with simulated physiological and vital signs such as the SIMMAN® simulator. Because the present teachings do not require a phantom to have any internal structure, a manikin can be easily used for the purposes of ultrasound imaging simulation.

One aspect of this system is the ability to quickly download image training volumes to a computer over the internet, described further below. In previous simulators, only a limited number of image volumes have been made available due in part to the technical problems with distributing such large files. In one embodiment, the image training volumes can be downloaded from the Internet using a very effective form of image compression, or be available on CD or DVD, likewise using a very effective form of image compression, such as an implementation of MPEG-4 compression.

Downloading the image volumes from the Internet may require special algorithms and software, which give computationally efficient and effective image compression. In this scheme image planes at sequential spatial locations are recorded as an image time sequence (series of image frames) or image loop; therefore, the compression scheme for a moving image sequence can be used to record a 3-D image volume. One codec in particular, H.264, can provide a compression ratio of better than 50 for moving images, while retaining virtually original image quality. In practice this means that an image volume containing one hundred frames can be compressed to a file only a few MBs in size. With a cable modem connection, such a file can be downloaded quickly. Even if the image volumes are stored on CD or DVD, image compression permits far more data storage. The codecs and their parameter adjustments will be selected based on their clinical authenticity. In other words, image compression cannot be applied without verifying first that important diagnostic information is preserved.

A library of ultrasound image training volumes may be developed, with a “sub-library” for each of the medical specialties that use ultrasound. Each sub-library will need to include a broad selection of pathologies, traumas, or other bodies of interest. With such libraries available the sonographer can stay current with advancing technology, and become well-experienced in his/her ability to locate and diagnose pathologies and/or trauma. The image training material may consist of 3-D image volumes—that is, it is composed of a sequence of individual scan frames. The dimensions of the scan frames can be quantified, either in distances or in round-trip travel times, as well as the spacing and spatial orientation of the individual scan planes. The image training material may also consist of a 3D anatomical atlas, which is treated by the ultrasound training system as if it were an image volume.

The image training volumes may be of two types: (i) static image volumes; and (ii) dynamic image volumes. A static image volume is generated by sweeping the transducer over a stationary part of a body and does not exhibit movement due to the heart and respiration. In contrast, a dynamic volume includes the cardiac generated movement of organs. For that reason it would appropriately be called a 4-D volume where the 4th dimension is time. In the 4-D case, the spatial locations of the scan planes are the same and are recorded at different times, usually over one cardiac cycle. For example, for 4-D imaging of the heart the time span will be equal to one cardiac cycle. The total acquisition time for each 3-D set in a 4-D dynamic volume set is usually small compared with the time for a complete cycle. A dynamic image volume will typical consist of 20-30 3-D image volumes, acquired with constant time interval over one cardiac cycle.

The image training volumes in the library/sub-libraries may be indexed by many variables: the specific anatomy being scanned; whether this anatomy is normal or pathologic or has trauma; what transducer type was used; and/or what transducer frequency was used, to name a few. Thus, one may have hundreds of image volumes, and such an image library may be built up over some time.

The training system provides an additional important feature: it can evaluate to what extent the sonographer has attained needed skills. It can track and record mock transducer movements (scan patterns) made to locate a given organ, gland or pathology, and it can measure how long it took the operator to do so. By touch screen annotation, the operator/trainee can identify the image frame that shows the pathology to be located. In another exercise, for example, although not limited thereto, the sonographer may be presented with ten image volumes, representing ten different individual patients, and be asked to identify which of these ten patients have a given type of trauma (e.g., abdominal bleeding, etc.), or a given type of pathology (e.g., gallstones, etc.).

The value of the virtual interactive training system is greatly increased by enabling the system to demonstrate that the student has improved his/her scanning ability in real-time, which will allow the system to be used for earning Continuing Medical Education (CME) credits. With touch screen annotation or another interactive method, the user can produce an overlay to the image that can be judged by the training system to determine whether a given anatomy, pathology or trauma has been located. The user may also be asked to determine certain distances, such as the biparietal diameter of a fetal head. Inferences necessary for diagnosis can also be evaluated, including the recognition of a pattern, anomaly or a motion.

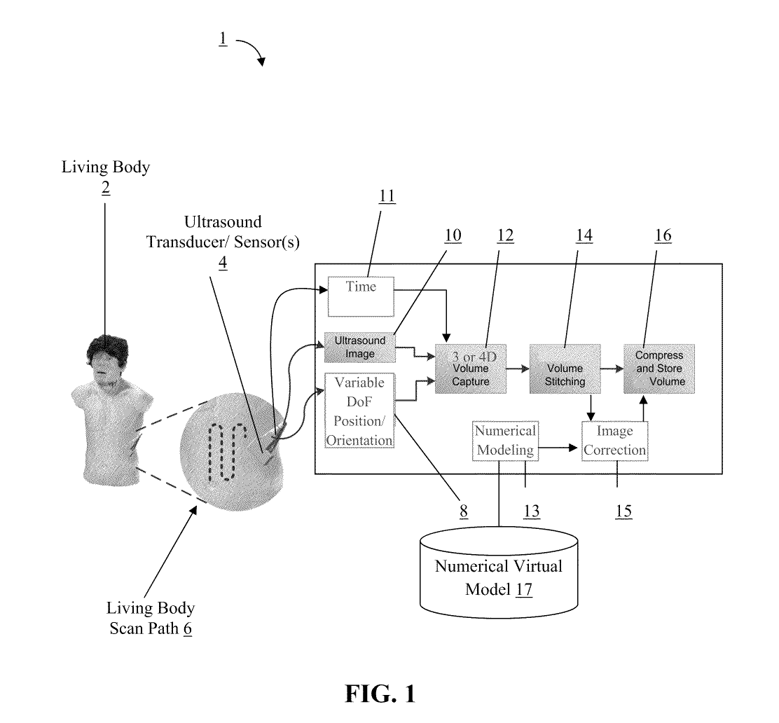

Referring to FIG. 1, shown is a pictorial depicting one embodiment of the method of generating ultrasound training image material. The ultrasound training image material is in the form of 3-D composite image volumes which are acquired from any number of living bodies 2. To be useful for training purposes, the training material should cover a significant segment of the human anatomy, such as, although not limited thereto, the complete abdominal region, a total neck region, or the lower extremity between hip and knee. A library of ultrasound image volumes can being assembled using many different living bodies 2. For example, although not limited thereto, humans having varying types of pathologies, traumas, or anatomies (collectively positions of interest) could be scanned in order to help provide diagnostic training and experience to the system operator/trainee. Any number of animals could also be scanned for veterinarian training. In addition, a healthy human could be scanned to create a 3-D image volume and one or more ultrasound scans containing some predetermined body of interest (e.g., trauma, pathology, etc.) could then be inserted, discussed further below.

Due to the size of the ultrasound transducer 4, a complete ultrasound scan of the living body 2 cannot be acquired in a single sweep. Instead, the scan path 6 will comprise multiple sweeps over the living body 2 being scanned. To aid in stitching separate 3-D ultrasound scans acquired using this freehand imaging approach into a single image volume, discussed further below, tracking sensors are used with the ultrasound transducer 4 to track its position and orientation 126. This may be done in 6 degrees of freedom (“DoF”), although not limited thereto. In such a way, each ultrasound image 10 of the living body 2 corresponds with position and orientation 126 information of the transducer 4. Alternatively, a mechanical fixture can be used to translate the transducer 4 through the imaging sequence in a controlled way. In this case, tracking sensors are not needed and image planes are spaced at uniform known intervals.

Because the individual ultrasound images 10 will be combined into a single 3-D image volume 12, it is helpful if there are no gaps in the scan path 6. This can be accomplished by at least partially overlapping each scan sweep in the scan path 6. A stand-off pad may be used to minimize the number of overlapping ultrasound to scans. Since the position and orientation 126 of the ultrasound transducer 4 is also recorded, any redundant scan information due to overlapping sweeps can be removed when the ultrasound images 10 are volume stitching 14, discussed further below.

Once the ultrasound images 10 are captured in a 3-D or 4-D (also using time 11) image volume 12, any overlaps or gaps in the scan pattern 6 can be fixed by using the position and orientation 126 during volume stitching 12. In 3-D, stitching can prove difficult to do manually. Conventional software can be used to stitch the individual ultrasound images 10 into complete 3-D volumes which completely representing the living body 2. The conventional software can line up the scans based on the recorded position and orientation 126. The conventional software can also implement a modified scanning process designed for multiple sweep acquisition, called ‘multi-sweep gated’ mode. In this mode, recording starts when the probe has been held still for about a second and stops when the probe is held still again. When the probe is lifted up and moved over, then held still again, another sweep is created and recording resumes. This can be repeated for any number of sweeps to form a multi-sweep volume, thus avoiding having to manually specify the extents of the sweeps in the post-processing phase. Alternatively, the acquired image planes of each sweep can be corrected for position and angle and interpolated to form a regularized 3D image volume that consists of the equivalent of parallel image planes.

Carrying out ultrasound image 10 acquisitions from actual human subjects presents several challenges. These arise from the fact that it is not sufficient to simply translate, rotate and scale one image volume to make it align with an adjacent one (affine transformation) in order to accomplish 3-D image volume stitching 14. The primary source of difficulties is motion of the body and organs due to internal movements and external forces. Internal movements are related to motion within the body during scanning, such as that caused by breathing, heart motion and intestinal gas. This causes relative deformation between scans of the same area. As a consequence, during 3-D image volume stitching 14 such areas do not line up perfectly, even though they should, based on position and orientation 126. External forces include irregular ultrasound transducer 4 pressure. When probe pressure is varied during the sweep, for example when the transducer is moved over the body, internal organs are compressed to different degrees, especially near the skin surface. Scan sweeps in different directions may also push organs in slightly different ways, further altering the ultrasound images 10. Thus, distortion due to varying ultrasound transducer 4 pressure presents the same type of alignment challenges as do the distortion due to internal movements.

3-D image volume stitching 14 can be accomplished first based on position and orientation 126 alone. Within and across ultrasound images 10 plane, registration based on similarity measures can be used in the overlap areas to determine regions that have not been deformed due to either internal or external forces. A fine degree of affine transformation may be applied to such regions for an optimal alignment, and such regions can serve as ‘anchor regions.’ For 4-D image volumes (including time 11), a sequence of moving images can be assembled where each image plane is a moving sequence of frames.

Most of the methods of registration use some form of a comparison-based approach. Similarity measures are typically statistical comparisons of two values, and a number of different similarity measures can be used for comparison of 2-D images and 3-D data volumes, each having their own merits and drawbacks. Examples of similarity measures are: (i) sum of absolute differences, (ii) sum-squared error, (iii) correlation ratio, (iv) mutual information, and (v) ratio image uniformity.

Regions adjacent to ‘anchor regions’ need to be aligned through higher degrees of freedom alignment processes, which also permits deformation as part of the alignment process. There are several such methods, such as twelve degree of freedom alignment. This involves aligning two images by translation, rotation, scaling and skewing. Following the affine alignment, a free-form deformation is performed to non-rigidly align the two images. For both of these alignments the sum of squared difference similarity measure may be used.

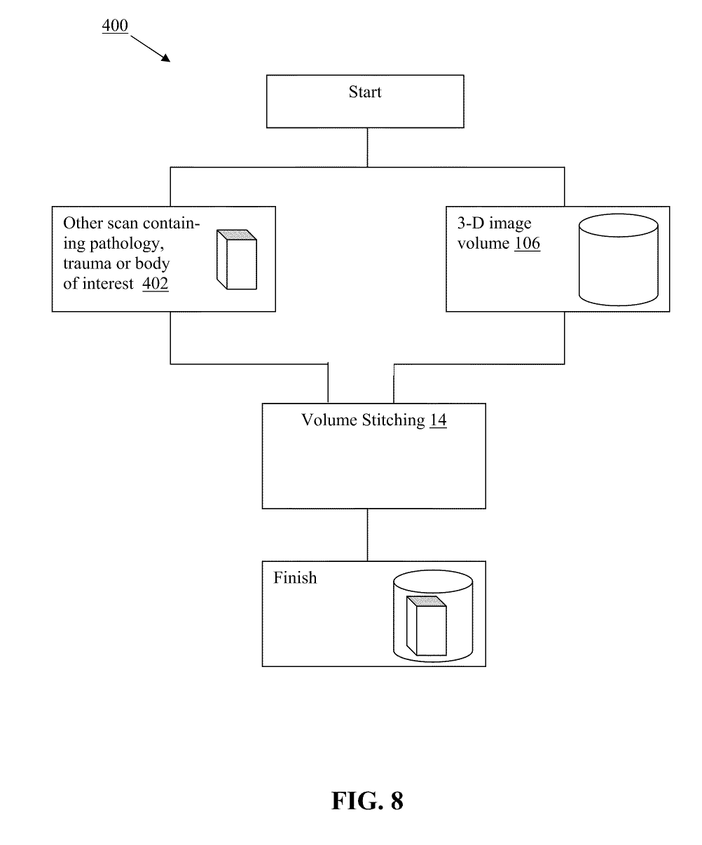

Whether dealing with a composite healthy image volume or a composite pathology or trauma image volume (FIG. 8, described further herein), the last processing step is an image volume scaling to make the acquired composite (stitched) image volume match in physical dimensions to the dimensions of the particular manikin in use. Using a numerical virtual model 17 and numerical modeling 13, image correction 15 scales and sizes the combined, stitched volume to match the dimensions of the manikin for virtual scanning. Image correction 15 may also correct inconsistencies in the ultrasound images 10 such as when the transducer 4 is applied with varying force, resulting in tissue compression of the living body 2.

Once the 3-D image volume stitching 14 and image correction 15 is complete, the training volume can be compressed and stored 16 in a central location. The composite, stitched 3-D volume can be broken into mosaics for shipping. Each mosaic tile can be a compressed image sequence representing a spatial 3-D volume. These mosaic tiles can then be uncompressed and repackaged locally after downloading to represent the local composite 3D volume.

Referring now to FIG. 2A, shown is a pictorial depicting one embodiment of the ultrasound training system. The system is designed to be an inexpensive, computer-based training system, in which the trainee/operator “scans” a manikin 20 using a mock transducer 22. The system is not limited to use with a lifelike manikin 20. In fact, “dummy phantoms” with varying attributes such as shape or size could be used. Because the 3-D image volumes 106 are stored electronically, they can be rescaled to fit manikins of any configuration. For instance, the manikin 20 may be hollow and/or collapsible to be more easily transported. A 2-D ultrasound image is shown on a display 114, generated as a “slice” of the stored 3-D image volume 106. 3D volume rendering, modified for faster rendering of voxel-based medical image volumes, is adjusted to display only a thin slice, giving the appearance of a 2-D image. Additionally, orthographic projection is used, instead of a perspective view, to avoid distortion and changes in size when the view of the image is changed. The “slicing” is determined by the mock transducer's 22 position and orientation in a preselected number of degrees of freedom relative to the manikin 20. The 3-D image volume 106 has been associated with the manikin 20 (described above) so that it corresponds in size and shape. As the mock transducer 22 traverses the manikin 20, the position and orientation permit “slicing” a 2-D image from the 3-D image volume 106 to imitate a real ultrasound transducer traversing a real living body.

Based on the selected 3-D image volume 106, the ultrasound image displayed may represent normal anatomy, or exhibit a specific trauma, pathology, or other physical condition. This permits the trainee/operator to practice on a wide range of ultrasound training volumes that have been generated for the system. Because the presented 2-D image will be derived from a pre-stored 3D image volume 106, genuine ultrasound scanner equipment is not needed. The system can simulate a variety of ultrasound scanning equipment such as different transducers, although not limited thereto. Since an ultrasound scanner is not needed and since the patient is replaced by a relatively inexpensive manikin or manikin 20, the system is inexpensive enough to be purchased for training at clinics, hospitals, teaching centers, and even for home use.

The mock transducer 22 uses sensors to track its position in training scan pattern 30 while it “scans” the manikin 20. Commercially available magnetic sensor may be used that dynamically obtain the position and orientation information in 6 degrees of freedom (“DoF”). All of these tracking systems are based on the use of a transmitter as the external reference, which may be placed inside or adjacent to the surface of the manikin. Magnetic or optical 6 DoF tracking systems will subsequently be referred to as external tracking systems.

For a PC-based simulation system, the tracking system represents in the order of ⅔ of the total cost. In order to overcome the complexity and expense of external tracking systems, the mock transducer 22 may use optical and MEMS sensors to track its position and orientation in 5 DoF relative to a start position. The optical system tracks the mock transducer's 22 position on the manikin 20 surface in two orthogonal directions, while the MEMS sensor tracks the orientation of the mock transducer 22 along three orthogonal coordinates. Both 5 DoF and 6 DoF of this type are very suitable for this system.

This tracking system does not need an external reference (transmitter) as a reference, but uses the start point and the start orientation as the reference. This type of system will be referred to as a self-contained tracking system. Nonetheless, registration of the position and orientation of the mock transducer 22 to the image volume and to the manikin 20 is necessary. Thus, the manikin 20 will need to have a reference point, to which the mock transducer 22 needs to be brought and held in a prescribed position before scanning can start. Due to drift, especially in the MEMS sensors, recalibration will need to be carried out with regular intervals, discussed further below. An alert may tell the training system operator when recalibration needs to be carried out.

As the training system operator “scans” the manikin 20 with the mock transducer 22, the position and orientation information is sent to the 3-D image slicing software 26 to “slice” a 2-D ultrasound image from the 3-D image volume 106. The 3-D image volume 106 is a virtual ultrasound representation of the manikin 20 and the position and orientation of the mock transducer 22 on the manikin 20 corresponds to a position and orientation on the 3-D image volume 106. The sliced 2-D ultrasound image shown on the display 114 simulates the image that a real transducer in that position and orientation would acquire if scanning a real living body. As the mock transducer 22 moves in relation to the manikin 20, the image slicing software 26 dynamically re-slices the 3-D image volume 106 into 2-D images according to the mock transducer's 22 position and orientation and shows them in real-time the display 114. This simulates the ultrasound scanning of a real ultrasound machine used on a living body.

Referring now to FIG. 2B, an embodiment of the present teachings is shown in which virtual subject 462 is displayed, for example, on the same display 114 as 2D ultrasound image 464 of virtual subject 462.

Referring now to FIG. 2C, a 3D image data representing a specific anatomy or pathology is drawn from an image training library 106 and combined with unique virtual subject appearance. As the trainee scans virtual subject 462 with mock transducer 22 on scan pad 460, anatomical and pathology identification and scan path analysis systems 466 provide 2D ultrasound image 464 based on the particular pathology selected.

Referring now to FIG. 2D, details of scan pad 460 and mock transducer 22 are shown in which scan pad 460 includes built-in position sensing, and mock transducer 22 includes MEMS-based gyro, giving three DoF angle sensing capabilities. Connecting transducer 22 to a computing processor, for example, training system processor 101, is transducer cable 468 providing 3 DoF orientation information of the mock transducer. Likewise, connecting scan pad 460 to training system processor 101 is scan pad cable 470 providing position information of mock transducer 22 relative to scan pad 460 to training system processor 101.

Referring now to FIG. 2E, scan pad 472 without built-in position sensing is shown along with mock transducer 22 with optical position sensing and MEMS-based angle sensing capabilities. Mock transducer 22 can include a three DoF MEMS gyro for angle sensing and an optical tracking sensor for position sensing. The optical tracking sensor may be a single sensor or a dual sensor with dual optical tracking elements 474. Transducer cable 468 can provide position and orientation information of the mock transducer relative to the scan pad. The configuration shown in FIG. 2E also includes optical tracking using the dot pattern tracking previously disclosed.

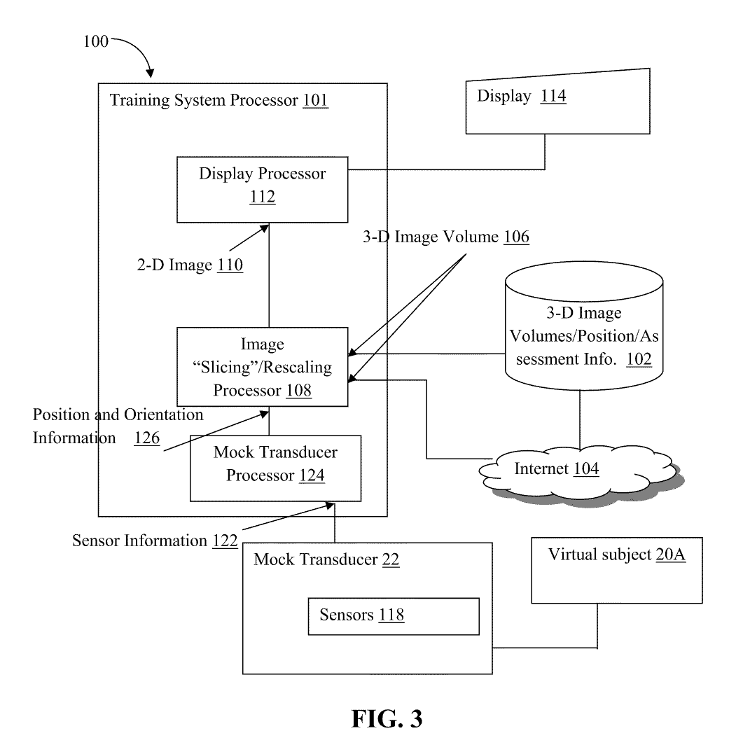

Referring now to FIG. 3, shown is a block diagram describing another embodiment of the ultrasound training system 100. 3-D image Volumes/Position/Assessment Information 102 containing trauma/pathology position and training exercises are stored on electronic media for use with the training system 100. 3-D image Volumes/Position/Assessment Information 102 may be provided over any network such as the Internet 104, by CD-ROM, or by any other adequate delivery method. A mock transducer 22 has sensors 118 capable of tracking the mock transducer's 22 position and orientation 126 in 6 or fewer DoF. The mock transducer's 22 sensor information 122 is transmitted to a mock transducer processor 124, which translates the sensor information 122 into mock position and orientation information. Sensors 118 can capture data using a compliant scan pad and a virtual subject 20A, the data resulting from either a scan pad, for example, a scan pad to capture the linear data, and a MEMS gyro in the mock transducer to capture angular data, or from an optical tracker in the mock transducer to capture the linear data, and MEMS gyro in the mock transducer to capture the angular data. As shown, this embodiment produces two images on display 114 (or on separate displays), the virtual subject with the virtual transducer (which moves in accordance with the movement of the mock transducer), and the ultrasound image corresponding to the virtual subject and the position of the virtual transducer.

The image slicing/rescaling processor 108 uses the mock position and orientation information to generate a 2-D ultrasound image 110 from a 3-D image volume 106. The slicing/rescaling processor 108 also scales and conforms the 2-D ultrasound image to the manikin 20. The 2-D image 110 is then transmitted to the display processor 112 which presents it on the display 114, giving the impression that the operator is performing a genuine ultrasound scan on a living body.

The position/angle sensing capability of the image acquisition system 1 (FIG. 1), or a scribing or laser scanning device or equivalent can be used to digitize the unperturbed manikin surface 21 (FIG. 2A). The manikin 20 can be scanned in a grid by making tight back-and-forth motions, spaced approximately 1 cm apart. A secondary, similar grid oriented perpendicular to the first one can provide additional detail. A surface generation script generates a 3-D surface mapping of the manikin 20, calculates an interpolated continuous surface representation, and stores it on a computer readable medium as a numerical virtual model 17 (shown on FIG. 1).

When a numerical virtual model 17 (shown on FIG. 1) has been generated, the 3D image volume 106 is scaled to completely fill the manikin 20. Calibration and sizing landmarks are established on both the living body 2 (FIG. 1) and the manikin 20 and a coordinate transformation maps the 3D image volume 106 to the manikin 20 coordinates using linear 3 axis anisotropic scaling. Only near the manikin surface 21 (FIG. 2A) will non-rigid deformation be needed.

For a mock transducer 22 having a self contained tracking system with less than 6 DoF, the a priori information of the numerical virtual model 17 (shown on FIG. 1) of the manikin surface 21 (FIG. 2A) can be used to recreate the missing degrees of freedom. The manikin surface 21 (FIG. 2A) can be represented by a mathematical model as S(x,y,z). Polynomial fits or non-uniform rational B-splines can be used for the surface modeling, for example. Calibration references points are used on the manikin 20 which are known absolutely in the image volume coordinate system of the numerical virtual model 17 (shown on FIG. 1). The orientation of the image plane and position of the mock transducer 22 sensors 118 are known in the image coordinate system at a calibration point. The local coordinate system of the sensor, if optical, senses the traversed distance from an initial calibration point to a new position on the surface. This distance is sensed as two distances along the orthogonal axes of the sensor coordinates, u and v. These distances correspond to orthogonal arc lengths, lᵤ and lᵥ along the surface. Each arc length lᵤ can be expressed as:

$${l}_{u} = {\int }_{a}^{x} \sqrt{[ 1 + {( \frac{\partial S}{\partial u} )}^{2} ]} d u$$

where S is the surface model, a is the x coordinate of the calibration start point, and x is the x coordinate of the new point, both in the image volume coordinate system. Because the arc length is measured, this equation can be solved iteratively for the x. Similarly, the arc length along the y axis lᵥ can be used to find y. The final coordinate of the new point, z, can be found by inserting x and y into the surface model S. The new known point replaces the calibration point and the process is repeated for the next position. The attitude of the mock transducer 22 in terms of the angles about the x, y, and z axes can be determined from the divergence of S evaluated at (x,y,z), if the transducer is normal to the surface, or from angle sensors. The relationship among the coordinate systems is described further below.

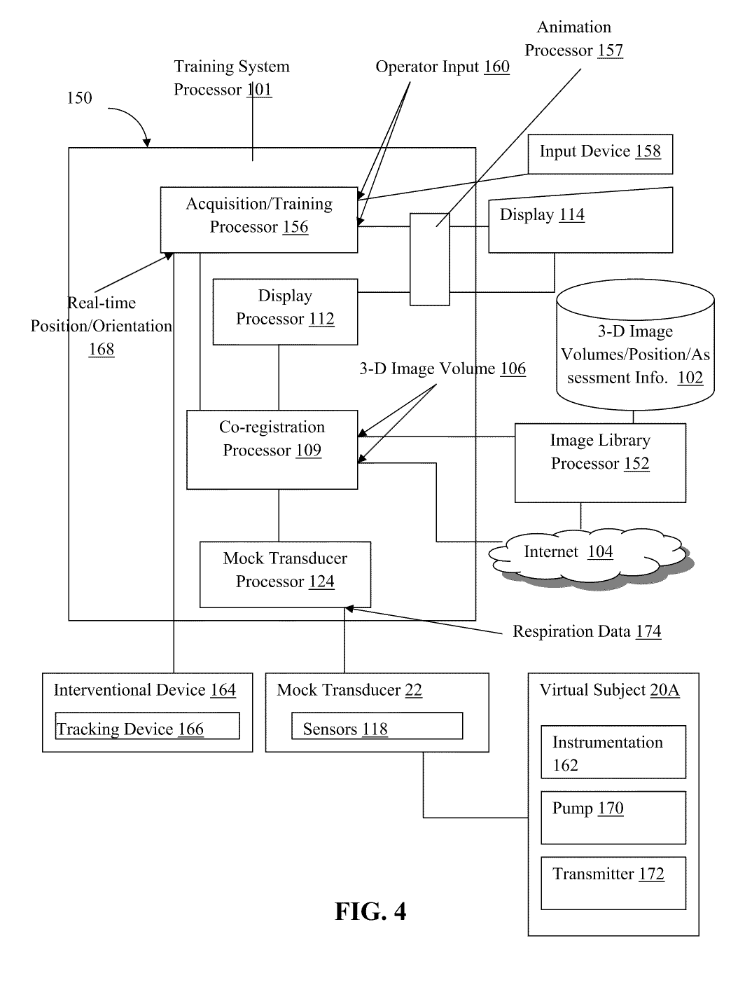

Referring now to FIG. 4, shown is a block diagram describing yet another embodiment of the ultrasound training system 150. FIG. 4 is substantially similar to FIG. 3 in that it uses a display 114 to show 2-D ultrasound images “sliced” from a 3-D image volume 106 using the mock transducer 22 position and orientation information. Also shown is an image library processor 152 which provides access to an indexed library of 3-D image volumes/Position/Assessment Information 102 for training purposes. A sub-library may be developed for any type of medical specialty that uses ultrasound imaging. In fact, the image volumes can be indexed by a variety of variables to create multiple libraries or sub-libraries based on, for example, although not limited thereto: the specific anatomy being scanned; whether this anatomy is normal or pathologic or has trauma; what transducer type was used; what transducer frequency was used, etc. Thus, as the size and diversity of the training system user group expands, there will be a need for many image volumes, and such an image library and sub-libraries will need to be built up over some time.

An important part of the training system is the ability to assess an operator's skills, discussed further below. Specifically, the training system can offer the following training and assessment capabilities: (i) it can identify whether the trainee operator has located a pertinent trauma, pathology, or particular anatomical landmarks (body of interest or position of interest) which as been a priori designated as such; (ii) it can track and analyze the operator's scan pattern 160 for efficiency of scanning by accessing optimal scan time 258; (iii) it allows an ‘image save’ feature, which is a common element of ultrasound diagnostics; (iv) it measures the time from start of the scanning to the diagnostic decision (whether correct decision or not); (v) it can assess improvement in performance from the scanning of the first case to the scanning of the last case by accessing assessment questions 260; and (vi) it can compare current scans to benchmark scans 256 performed by expert sonographers.

The 3-D image volumes/Position/Assessment Information 102 stored on electronic media has learning assessment information, for example, benchmark scan patterns and optimal times to identify bodies of interest, associated with the ultrasound information. The training system can determine the approximate skill level of the sonographer in scanning efficiency and diagnostic skills, and—after training—demonstrate the sonographer's improvement in his/her scanning ability in real-time, which will allow the system to be used for earning CME Credits. One indicator of skill level is the operator's ability to locate a predetermined trauma, pathology, or abnormality (collectively referred to as “bodies of interest” or “position of interest”). Any given image volume for training may well contain several bodies of interest. Other training exercises are possible, such as where the sonographer is presented with several image volumes, say ten image volumes, representing 10 different individual patients, and is asked to identify which of these ten patients have a given type of trauma such as abdominal bleeding, or a given type of pathology such as gallstones.

A co-registration processor 109 co-registers the 3-D image volume 106 with the surface of the manikin 20 in a predetermined number of degrees of freedom by placing the mock transducer 22 at a calibration point or placing a transmitter 172 inside said manikin 20. A training processor 156 can then compare the operator's training scan, determined by sensors 118, against, for example, a benchmark ultrasound scan. The training processor 156 could compare the operator's scan with a benchmark scan pattern and overlap them on the display 114, or compare the time it takes for the operator to locate a body of interest with the optimum time. The operator's scan path can be shown on a display 114 with a representation of the numerical virtual model 17 (FIG. 1) of the manikin 20. If instrumentation 162 or a pump 170 is used with the manikin 20 in order to produce artificial physiological life signs data 174 such as respiration, discussed further below, an animation processor 157 may provide animation to the display 114. The pump 170 may be used with an inflatable phantom to enhance the realism of respiration with a rescaling processor dynamically rescaling the 3-D ultrasound image volume to the size and shape of the manikin as it is inflated and deflated.

An interventional device 164, such as a mock IV needle, can be fitted with a 6 DoF tracking device 166 and send real-time position/orientation 168 to the acquisition/training processor 156. This permits the trainee operator to practice other ultrasound techniques such as finding a vein to inject medicine. Using the position/orientation 168, the animation processor 157 can show the simulation of the needle injection position on the display 114.

If a touch screen display is used, the trainee can indicate the location of a body of interest by circling it with a finger or by touching its center, although not limited thereto. If a regular display 114 is used, then another input device 158 such as a mouse or joystick may be used. The training processor 156 can also determine whether a given pathology, trauma, or anatomy has been correctly identified. For example, it can provide a training goal and then determine whether the user has accomplished the goal, such as correctly locating kidney stones; liver lesions, free abdominal fluid, etc. The operator may also be asked to determine certain distances, such as the biparietal diameter of a fetal head. Inferences necessary for diagnosis such as the recognition of a pattern and anomaly or a motion can also be evaluated.

The scan path, that is, the movement of the mock transducer 22 on the surface of the manikin 20, can be recorded in order to assess scanning efficiency over time. The effectiveness of the scanning will be very dependent on each diagnostic objective. For example, expert scanning for the presence of gallstone will have a scan pattern that is very different from the expert scanning to carry out a FAST (Focused Abdominal Sonography for Trauma) exam to locate abdominal free fluid. The training system can analyze the change in time to reach a correct diagnostic decision over several training sessions (image volumes and learning assessment information 154), and similarly the development of an effective scan pattern. Scan paths may also be shown on the digitized surface of the manikin 20 rendered on the display 114.

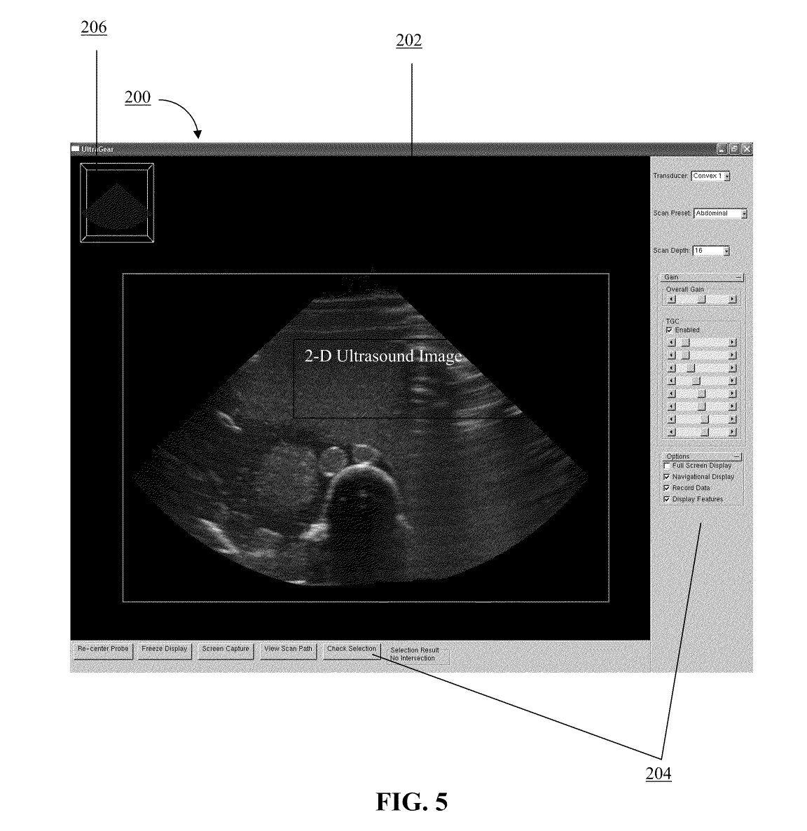

Referring now to FIG. 5, shown is pictorial depicting one embodiment of the graphical user interface (“GUI”) imaging system control panel 200 for the display of the ultrasound training system. The GUI tries to make the training session as realistic as possible by showing a 2-D ultrasound image 202 in the main window and associated ultrasound controls 204 on the periphery. As discussed above, the 2-D ultrasound image 202 shown in the GUI is updated dynamically based on the position and orientation of the mock transducer scanning the manikin. A navigational display 206 can be observed in the upper left hand corner, which shows the operator the location of the current 2-D ultrasound image 202 relative to the overall 3-D image volume.

Miscellaneous ultrasound controls 204 add to the degree of realism on an image, such as focal point, image appearance based on probe geometry, scan depth, transmit focal length, dynamic shadowing, TGC and overall gain. All involve modification of the 2-D ultrasound image 202. In addition, the user can choose between different transducer options and between different image preset options. For example, the GUI may have ‘Probe Re-center’ and ‘freeze display’ and record options. The emulation of overall gain and time gain control (TGC) allow the user to control the overall image brightness and the image brightness as a function of range. For TGC, the scan depth is divided into a number of zones, typically eight, the brightness of which is individually controllable; linear interpolation is performed between the eight adjustment points to create a smooth gradation. The overall gain control is implemented by applying a semi-opaque mask to the image being displayed. This also means that the source image material needs to be acquired with as good a quality as possible; for example, multi-transmit splicing is employed whenever possible to maximize resolution.

Focal point implementation means that image presentation outside the selected transmit focal region is slightly degraded with an appropriate, spatially varying slight smoothing function. Image appearance based on probe geometry involves making modifications near the skin surface so that for a convex transducer the image has a radial appearance, for a linear array transducer it has a linear appearance, and for a phased array it has a pie-slice-shaped appearance. By applying a mask to the image being viewed, it can be altered to take on the appearance of the image geometry of the specific transducer. This allows users to experience scanning with different probe shapes and extends the usefulness of this training system. This masking can be accomplished using a ‘Stencil Buffer’. A black and white mask is defined which specifies the regions to be drawn or to be blocked. A comparison function is used to determine which pixels to draw and which to ignore. By appropriately drawing and applying the stencil, the envelope of the display can be made to take on any shape. Different stencils are generated based on the selected probe geometry, to accurately portray the viewing area of the selected probe.

Simulation of Time Gain Compensation (TGC) and absorption with depth provide user interaction with these controls. User control settings can be recorded and compared to preferred settings for training purposes. Dynamic shadowing involves introducing shadowing effect “behind” attenuating structures where “behind” is determined by the scan line characteristics of the particular transducer geometry that is being emulated.

By using a finger or stylus on a touch screen or a mouse, trackball, or joystick on a regular screen, the operator can locate on the displayed image specific bodies of interest that may represent a specified trauma, pathology or abnormality training purposes. The training system can verify whether the body of interest was correctly identified, and permits image capture so that the operator has the opportunity to view and play back the entire scan path.

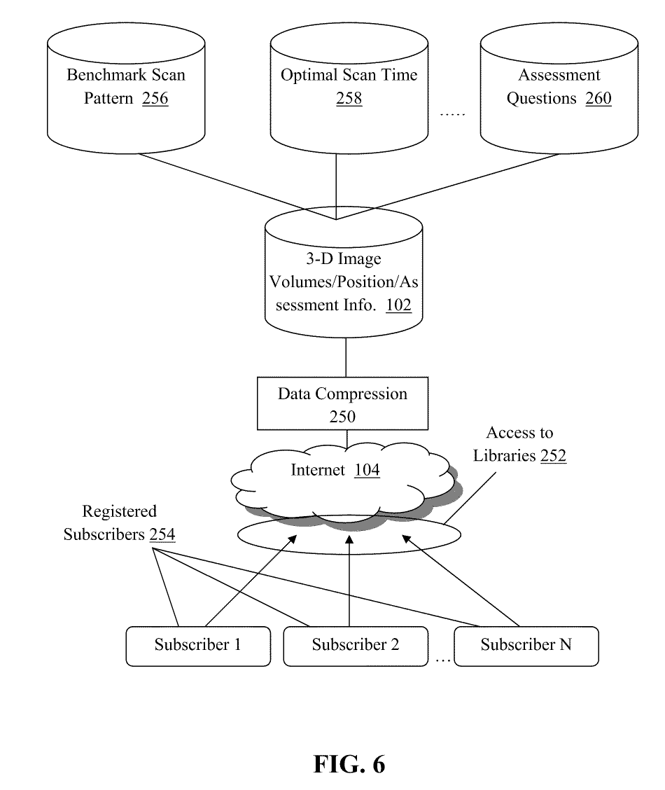

Referring now to FIG. 6, shown is a block diagram describing one embodiment of the method of distributing ultrasound training material. The 3-D ultrasound image volumes and training assessment information 102 may be distributed over a network such as the Internet 104. A central storage location allows a comprehensive image volume library to be built, which may have general training information for novices, or can be as specialized as necessary for advanced users. Registered subscribers 254 may locate pertinent image volumes by accessing libraries 252 where image volumes are indexed into sub-libraries by medical specialty, pathology, trauma, etc.

In order for an image library to be effective, it must be possible to quickly download the image volumes to the training computer over a network such as the Internet 104. To do so may require compression 250 which reduces the size of the downloadable files but retains adequate image quality. One promising codec for this is MPEG-4, part 10, also known as H.264. Use of H.264 has demonstrated that a compression ratio of 50:1 is realistic without discernable loss of image details. This means in practice that a composite image volume can be compressed to a file of maybe 5-10 MBs in size. With a cable modem connection, such a file can be downloaded in 5 to 10 seconds. The download and un-compression can be conveniently carried out using a decoding algorithm such as Apple's QuickTime.

A frame server can produce individual image frames for H.264 encoding. The resulting encoded bit stream will then either be stored to disk or transmitted over TCP/IP protocol to the training computer. A container format stores metadata for the bit stream, as well as the bit stream itself. The metadata may include information such as the orientation of each scan plane in 3-D space, the number of scan planes, the physical size of an image pixel, etc. An XML formatted file header for metadata storage may be used, followed by the binary bit stream.

For 4-D (including time) and/or Doppler image simulation having larger data sets, two methods can be used. 3D image volumes tagged with relative time of acquisition and are accessed using the same methods previously described for still imaging except that different memory locations are accessed in sequence and repeated according to increasing time tags. In a second method, the previous still methods are employed for stitching and the creation of a 3-D image volume of the first frame. These settings are then used to access a full 4-D data set that is derived from compressed image files (including time) at each spatial image plane location. Frames are cycled through the same set of display operations for a 2D image plane selected for visualization and display.

With such libraries available the sonographer can stay maintain his/her ability to locate and diagnose pathologies and/or trauma. Even if the image volumes are stored on CD or even DVD, image compression permits far more data storage. When a trainee/operator receives the image volumes from the centrally stored library, he or she would need to uncompress the image volume cases and placing them in memory of a computer for use with the training system. The training information downloaded would include not only the ultrasound data, but the training lessons, and simulated generic or specific diagnostic ultrasound system display configurations including image display and simulated control panels.

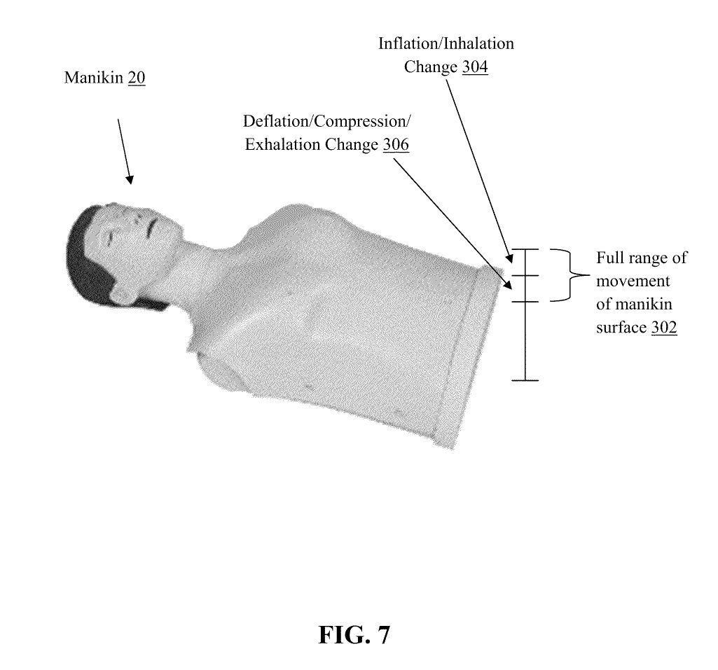

Referring now to FIG. 7, shown is a pictorial depicting one embodiment of the manikin or manikin 20 used with the ultrasound training system. To improve the degree of realism, the ultrasound training system may have as options the ability to simulate respirations or to account for compression of the phantom surface by the mock transducer. Simulated respiration or transducer compression will affect the manikin 20 surface and create a full range of movement 302. For instance, if the manikin 20 “exhales” by pumping air out and reducing the internal volume of air, the surface will experience a deflationary change 306. Similarly, if it “inhales” by pumping air in and increasing the internal air volume, the surface will experience an inflationary change 304. To increase the realism of the training system, any change of the manikin 20 surface should affect the ultrasound image being displayed since the mock transducer will move with the full range of movement 302 of the surface.

In order to add the realism of breathing, one of two methods can be employed. For the first method, the displacement of the skin surface at one of more points will need to be tracked, and if an external tracking system is used, this is easily done by mounting one or more sensors under the skin surface to measure the displacement. This information will then be used to dynamically rescale the image volume (from which the 2-D ultrasound image is “sliced”) so that so that it matches the shape and size of the manikin 20 at any point in time during the respiratory cycle. The image volume may be a 3-D ultrasound image volume, a 4-D image volume or a 3-D anatomical atlas.

A second method may be employed if an external tracking system is not used (the self-contained tracking system is used instead). This involves the acquisition of a 4-D image volume (e.g., several image volumes, each taken at intervals within a respiratory cycle). In this case, an appropriately sized and shaped 3-D image volume, according to the time during the respiratory cycle, is used for “slicing” a 2-D ultrasound image for display. The movement of the phantom surface for each point in time of the respiratory cycle must be determined a priori. The 3-D image volume can then be dynamically rescaled based on the time of the respiratory cycle, according to the known size and shape of the phantom at that point in the respiratory cycle.

Respiration can be emulated by the inclusion of a pump 170 (FIG. 4). A pumping system should be able to regulate the tidal volume and breathing rate. The ability to set a specific breathing pattern with corresponding dynamic image scaling will add a high degree of realism to the ultrasound training system. Controls for respiration may be included in the GUI or placed at a separate location on the training system.

During actual ultrasound scanning, the surface of the living body's skin can be compressed by pressing the transducer into the skin. This can also happen in training if a compressible phantom is being used. This type of image compression can be emulated with the ultrasound training system. If an external tracking system with 6 degrees of freedom is used, the degree of local compression is readily determined from the amount of displacement determined from a comparison of the mock transducer position/attitude to the digitized unperturbed surface of the manikin as stored in the numerical modeling. A rescaling processor may dynamically rescale the 2-D ultrasound image to the size and shape of the manikin as it is compressed by the mock transducer. A local deformation model can be developed to simulate the appropriate degree of local (near surface) image compression based on both numerically-calculated compression as well as shear stress distribution in the scan plane, based on approximate shear modulus values for biological soft tissue.

For tracking systems with 5 DoF (missing the vertical direction normal to the skin surface), the compression displacement cannot be measured directly. However, the force that the mock transducer applies to the phantom surface can be determined through the use of force sensors integrated into the mock transducer (placed inside the surface that makes contact with the phantom). The compliance of the phantom at each point on its surface can be mapped a priori. By combining the known location of the mock transducer on the surface of the phantom, the known compliance of the phantom at that point, and the applied force measured by pressure sensors, actual local compression can be calculated. The image deformation can then be made by appropriately sizing and shaping the image volume as discussed above.

An additional degree of realism can optionally be emulated by detecting whether an adequate amount of acoustic gel has been applied. This can most readily be done with electrical conductivity measurements. Specifically, the part of the sham transducer in contact with the “skin” of the manikin will contain a small number of electrodes (say three or four) equally spaced over the long axis of the transducer. In order for the ultrasound image to appear, the electrical conductivity between anyone pair of electrodes needs to be below a given set value determined by the particular gel in use.

In one embodiment of a recalibration system used to recalibrate the mock transducer, a transducer and 6 DoF sensor can be held in a clamp as shown exemplarily by P-W Hsu et al. in Freehand 3D Ultrasound Calibration: A Review, December 2007, FIG. 8(b) on page 9. The materials for the recalibration system can be selected to minimize interference with magnetic tracking systems using, for example, nonmagnetic materials. If the anatomical data of the phantom has been collected, it can be shown on the display.

A 6 DoF transformation matrix relates the displayed scan plane to the image volume. This matrix is the product of matrix 1, a transformation between the reconstruction volume and the location of the tracking transmitter and is used to remove any offset between the captured image volume and the tracking transmitter, matrix 2 is the transformation between the tracking transmitter and tracking receiver, which is what is determined by the tracking system, and matrix 3 is the transformation between the receiver position and the scan image. This last matrix is obtained after physically measuring the location of the imaging plane to movements along Dofs in a mechanical fixture.

Referring to FIG. 8, shown is a block diagram describing one embodiment of volume stitching system 400 for stitching ultrasound scans (also shown in FIG. 1). A particular challenge is the stitching of a 3-D image volume image from a patient with a given trauma or pathology (body of interest), into a 3-D image volume from a healthy volunteer. In this case, the first step will be to outline the tissue/organ boundaries inside the healthy image volume which correspond to the tissue/organ boundaries of the trauma or pathology image volume. This step may be done manually. Note that the two volumes probably will not be of the same size and shape. Next, the healthy tissue volume lying inside the identified boundaries will be removed and substituted with the trauma or pathology volume 402. Again, there may be unfilled gaps as well as overlapping regions after this substitution has been completed. Finally, a type of freeform deformation along with scaling, translation and rotation, will be applied to produce a realistic and continuous image volume. This allows pathology or trauma scans to be reused without fear of abusing ill patients by repeatedly scanning them or having to conduct a complete body scan.

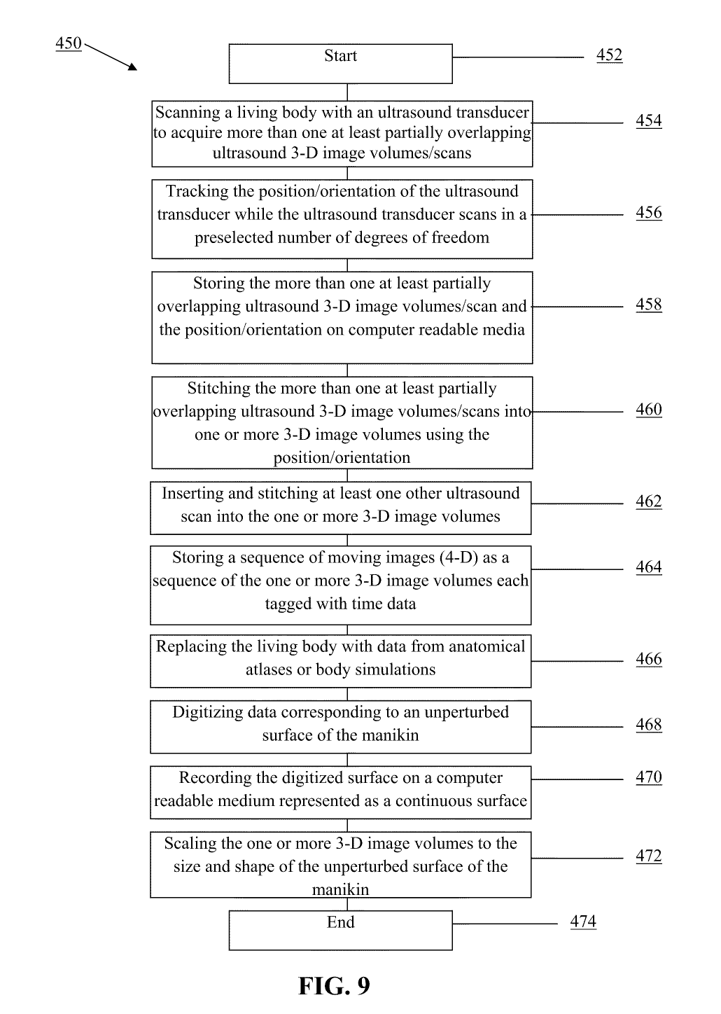

Referring now to FIG. 9, shown is a block diagram describing one embodiment of the method of generating ultrasound training image material. The following steps take place: Scanning a living body with an ultrasound transducer to acquire more than one at least partially overlapping ultrasound 3-D image volumes/scans 454; Tracking the position/orientation of the ultrasound transducer while the ultrasound transducer scans in a preselected number of degrees of freedom 456; Storing the more than one at least partially overlapping ultrasound 3-D image volumes/scan and the position/orientation on computer readable media 458; Stitching the more than one at least partially overlapping ultrasound 3-D image volumes/scans into one or more 3-D image volumes using the position/orientation 460; Inserting and stitching at least one other ultrasound scan into the one or more 3-D image volumes 462; Storing a sequence of moving images (4-D) as a sequence of the one or more 3-D image volumes each tagged with time data 464; Replacing the living body with data from anatomical atlases or body simulations 466; Digitizing data corresponding to an unperturbed surface of the manikin 468; Recording the digitized surface on a computer readable medium represented as a continuous surface 470; and Scaling the one or more 3-D image volumes to the size and shape of the unperturbed surface of the manikin 472.

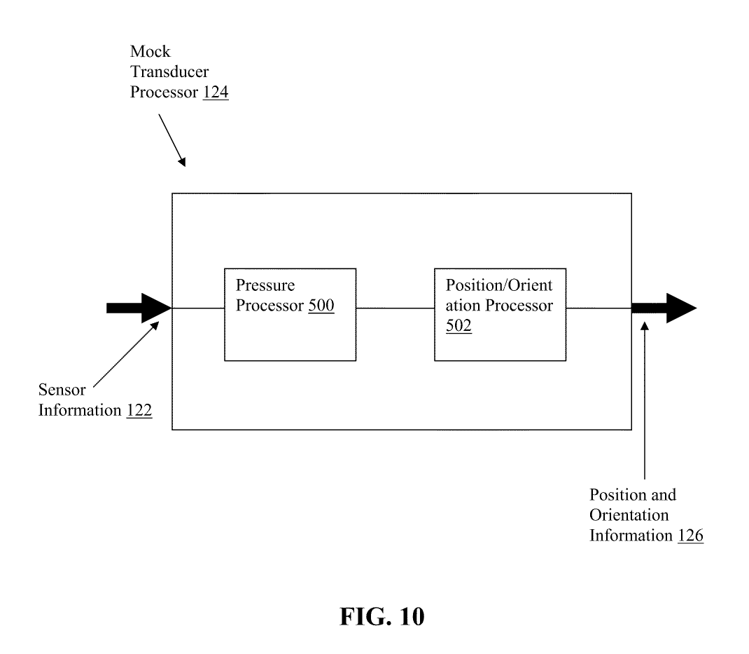

Referring now to FIG. 10, shown is a block diagram describing one embodiment of the mock transducer pressure sensor system. Sensor information 122 provided by sensors 118 in the mock transducer 22 (FIG. 3) is first relayed to the pressure processor 500, which, in one embodiment, receives information from a transmitter that is internal to manikin 20. The pressure processor 500 can translate the pressure sensor information and, together with data from the positional/orientation processor 502, can determine the degree of deformation of the manikin's surface, based on a pre-determined compliance map of the manikin. The deformation of the manikin's surface, thus indirectly measured, can be used to generate the appropriate image deformation in the image region near the mock transducer.

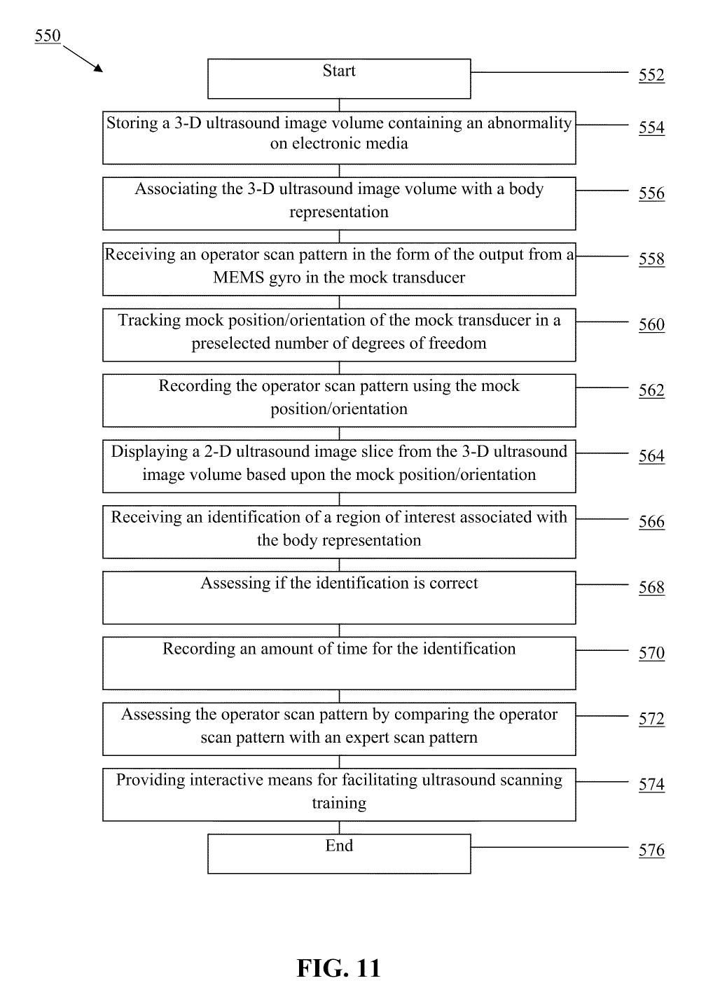

Referring now to FIG. 11, shown is a block diagram describing one embodiment of the method of evaluating an ultrasound operator. Throughout this specification, the term “body representation” refers to embodiments such as, but not limited to the physical manikin and the combination of scan surface and virtual subject. The method can include, but is not limited to including, the steps of storing 554 a 3-D ultrasound image volume containing an abnormality on electronic media, associating 556 the 3-D ultrasound image volume with a body representation, receiving 558 an operator scan pattern in the form of the output from the MEMS gyro in the mock transducer and the output from scan surface or optical tracking, tracking 560 mock position/orientation of the mock transducer (22) in a preselected number of degrees of freedom, recording 562 the operator scan pattern using the position/orientation, displaying 564 a 2-D ultrasound image slice from the 3-D ultrasound image volume based upon the position/orientation, receiving 566 an identification of a region of interest associated with the body representation; assessing 568 if the identification is correct, recording 570 an amount of time for the identification, assessing 572 the operator scan pattern by comparing the operator scan pattern with an expert scan pattern, and providing 574 interactive means for facilitating ultrasound scanning training.

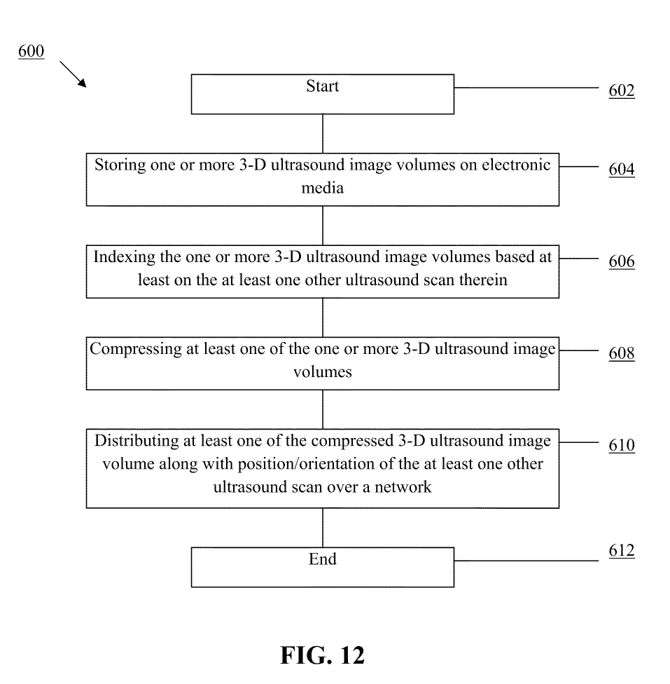

Referring now to FIG. 12, shown is a block diagram describing one embodiment of the method of distributing ultrasound training material. The method can include, but is not limited to including, the steps of storing 604 one or more 3-D ultrasound image volumes on electronic media, indexing 606 the one or more 3-D ultrasound image volumes based at least on the at least one other ultrasound scan therein, compressing 608 at least one of the one or more 3-D ultrasound image volumes, and distributing 610 at least one of the compressed 3-D ultrasound image volume along with position/orientation of the at least one other ultrasound scan over a network.

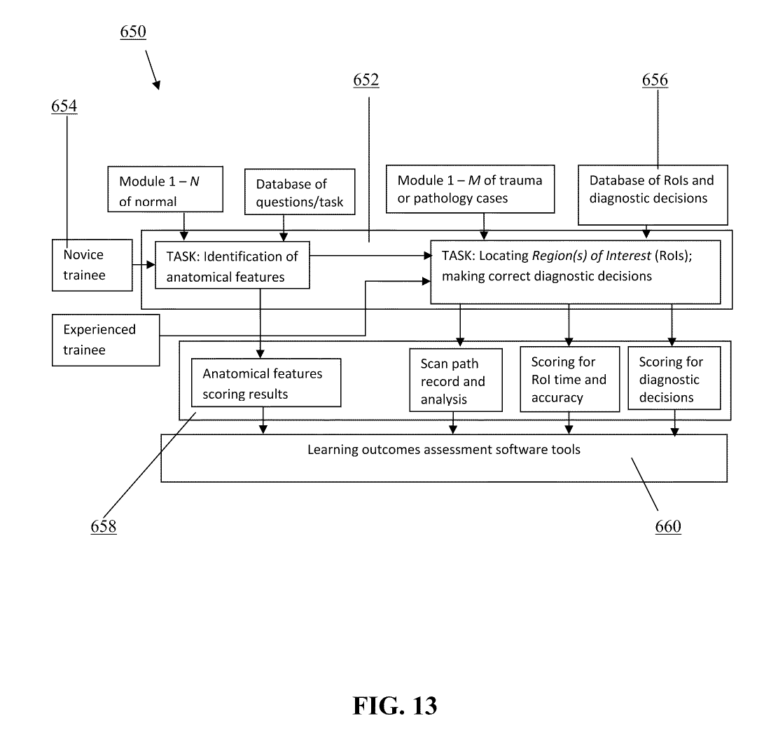

Referring now to FIG. 13, shown is a block diagram of another embodiment of the ultrasound training system. The instructional software and the outcomes assessment software tool have several components. Two task categories 652 are shown. One task category deals with the identification of anatomical features, and this category is intended only for the novice trainee, indicated by a trainee block 654. This task operates on a set of training modules of normal cases, numbered 1 to N, and a set of questions is associated with each module. The trainee will indicate the image location of the anatomical features and organs associated with the questions by circling the particular anatomy with a finger or mouse.

The other task category operates on a set of training modules of trauma or pathology cases, numbered 1 to M, and this category deals with a database 656 of the localization of a given Region of Interest (“RoI”, also referred to as “body of interest”). The trainee operator performs the correct localization of the RoI based on a set of clinical observations and/or symptoms described by the patient, made available at the onset of the scanning, along with the actual image appearance. In addition to finding the RoI, a correct diagnostic decision must also be given by the trainee. This task category is intended for the more experienced trainee, indicated with a trainee block. The source material for these two task categories 652 is given in the row of blocks at the top of FIG. 13. The scoring outcomes 658 of the tasks are recorded in various formats. The scoring outcomes 658 feed the scoring results into the learning outcomes assessment tools 660, which intend to track improvement in scanning performance, along different parameters.

A training module may contain a normal case or a trauma or pathology case, where a given module consists of a stitched-together set of image volumes, as described earlier. Each module has an associated set of questions or tasks. If a task involves locating a given Region of Interest (RoI), then that RoI is a predefined (small) subset of the overall volume; one may think of a RoI as a spherical or ellipsoidal image region that encloses the particular anatomy or pathology in question. The predefined 3-D volume will be defined by a specialist in emergency ultrasound, as part of the preparation of the training module.

The instructional software is likely to contain several separate components such as the development of an actual trauma or performing an exam effectively and accurately. The initial lessons may contain a theory part, which could be based on an actual published text, such as Emergency Ultrasound Made Easy, by J. Bowra and R. E. McLaughlin.

Four individual scoring outcomes 658 are identified in FIG. 13. One scoring system tracks the correct localization of anatomical features, possibly including the time to locate them. Another scoring system records the scan path and generates a scan effectiveness score by comparing the trainee's scan path to the scan path of an expert sonographer for the given training module. Another scoring system scores for diagnostic decision-making, which is similar to the scoring system for the identification of anatomical features.

Scoring for correct identification of the RoI, along with recoding of the elapsed time, is a critical component of trainee assessment. Verification that the RoI has been correctly identified is done by comparing the coordinates of the RoI with the coordinates of the region of the ultrasound image, circled by trainee on the touch screen. The detection system will be based on the Method of Collision Detecting of moving objects, common in computer graphics. Collision detection is applied in this case by testing whether the selection collides with or is inside the bounding spheres or ellipsoids. When the trainee has located the correct region of interest in an ultrasound image, the time and accuracy of the event is recorded and optionally given as feedback to the trainee. The scoring results over several sessions will be given as an input to the learning outcomes assessment software.

3-D anatomical atlases can be incorporated into the training material and will be processed the same way as the composite 3D image volumes. This will allow an inexperienced clinical person first to scan a 3D anatomical atlas, and here we can consider a 3D rendering with the 2D slice based on the transducer position highlighted.

Because of the technique that scales the image volume to the manikin surface, it can also be applied to retrofit the composite 3D image volume to an already instrumented manikin. An instrumented manikin has artificial life signs such as a pulse, EKG, and respiratory signals and movements available. Advanced versions also are used for interventional training to simulate an injury or trauma for emergency medicine training and life-saving intervention. The addition of ultrasound imaging provides a higher degree of realism. In this application, the ultrasound image volume(s) are selected to synchronize with the vital signs (or vice versa) and to aid in the diagnosis of injury as well as to depict the results of subsequent interventions.

While the present teachings have been described above in terms of specific embodiments, it is to be understood that they are not limited to these disclosed embodiments. Many modifications and other embodiments will come to mind to those skilled in the art to which these present teachings pertain, and which are intended to be and are covered by both this disclosure and the appended claims. It is intended that the scope of the present teachings should be determined by proper interpretation and construction of the appended claims and their legal equivalents, as understood by those of skill in the art relying upon the disclosure in this specification and the attached drawings.

We claim:

1. A method for generating ultrasound training image material, comprising the steps of:
scanning a living body with an ultrasound transducer to acquire more than one at least partially overlapping ultrasound 3D image volume/scan;
tracking transducer position and orientation of the ultrasound transducer while the ultrasound transducer scans in a preselected number of degrees of freedom;
storing the more than one at least partially overlapping ultrasound 3D image volumes/scan and the transducer position and the orientation on computer readable media; and
stitching the more than one at least partially overlapping ultrasound 3D image volume/scan into one or more 3D image volumes based on the position/orientation to form a library of the one or more 3D image volumes.

2. The method of claim 1 further comprising the step of:
storing a sequence of moving images as a sequence of the one or more 3D image volumes each tagged with time data.

3. The method of claim 1 further comprising the step of:
selecting, from the library, one of the one of more 3D image volumes;
associating the selected image volume with a body representation; and
presenting 2D image data based on position and orientation information from the mock transducer on the body representation and the selected 3D image volume.

4. The method of claim 1 further comprising the step of:
scaling the one or more 3D image volumes to the size and shape of a body representation.

5. The method of claim 1 further comprising the step of:
receiving the position and orientation information from the mock transducer;
generating 2D image data obtained from reslicing the selected 3D image volume based on the position and orientation information; and
displaying the 2D image data.

6. An image acquisition system comprising:
an ultrasound transducer and associated ultrasound imaging system;
at least one 6 degrees of freedom tracking sensor integrated with said ultrasound transducer/sensor;
a volume capture processor utilizing a position/orientation of each image frame relative to a reference point, to produce at least one 3-D volume; and
a volume stitching processor combining a plurality of said at least one 3-D volumes into one composite 3D volume.

7. The image acquisition system of claim 6 further comprising:
an image correction processor applying image correction to said ultrasound 3-D image volumes caused by tissue motion artifacts, resulting in said at least one composite 3D volume reflecting tissue motion correction.

8. The image acquisition system of claim 6 further comprising:
numerical model processor acquiring a numerical virtual model of a digitized surface of a body representation, and interpolating and recording said digitized surface, represented as a continuous surface, on a computer readable medium.

9. An ultrasound training system, comprising:
one or more scaled composite 3-D image volumes stored on electronic media, said one or more image volumes where said image volumes have been generated by combining individual 3D ultrasound image volumes recorded from a living body;
a body representation;
a 3-D composite image volume scaled to match the size and shape of said body representation;
a mock transducer having sensors for tracking a position and orientation of said mock transducer relative to said body representation in a preselected number of degrees of freedom;
an acquisition/training processor having computer code calculating a 2-D ultrasound image from said one or more composite image volumes based on said position and orientation; and
a display presenting said 2-D ultrasound image for training an operator.

10. The system of claim 9 wherein said sensors are selected from a group consisting of a MEMS gyro, a graphical tablet, an optical tracking device having at least one computer mouse, and an optical tracking device having a dot pattern.

11. The system of claim 9 wherein said acquisition/training processor comprises computer code configured to:
record a training scan pattern and a sequence of time stamps associated with the position and orientation, scanned by the operator, of said body representation on said electronic media based on said position/orientation;
compare a benchmark scan pattern, scanned by an experienced sonographer, of said body representation with said training scan pattern; and
store results of the comparison on said electronic media.

12. The system of claim 9 further comprising:
a co-registration processor co-registering said 3-D composite image volume with the surface of said body representation in 6 DOF by placing said mock transducer at a specific calibration point.

13. The system of claim 9 further comprising:
a co-registration processor co-registering said 3-D composite image volume with the surface of said body representation in 6 DOF by placing said mock transducer at a specific location on said body representation.

14. The system claim 9 further comprising:
a pressure processor receiving information from said sensors in said mock transducer.

15. The system of claim 14 further comprising:
a scaling processor scaling and conforming a numerical virtual model to the actual physical size of said body representation as determined by said digitized surface, and modifying a graphic image based on said information when a force is applied to said mock transducer and the surface of said body representation.

16. The system of claim 9 further comprising:
instrumentation associated with said body representation configured to produce artificial physiological life signs, wherein said display is synchronized to said artificial life signs, changes in said artificial life signs, and changes resulting from interventional training exercises.

17. The system of claim 9 further comprising:
a position/orientation processor calculating the 6 DoF mock position/orientation in real-time from a priori knowledge of said body representation and less than 6 DoF mock position/orientation on said body representation.

18. The system of claim 9 further comprising:
an interventional device fitted with a 6 DoF tracking device that sends real-time position/orientation to said acquisition/training processor.

19. The system of claim 9 further comprising:
a pump introducing artificial respiration to said body representation, said pump providing respiration data to a mock transducer processor) and inflating said body representation; and
an image slicing/rescaling processor dynamically rescaling said 3-D image volume to the size and shape of said body representation as said body representation is inflated.

20. The system of claim 19 further comprising:
an animation processor representing an animation of said interventional device inserted in real-time into said 3-D ultrasound image volume.

21. A method for evaluating an ultrasound operator comprising the steps of:
storing a 3-D ultrasound image volume containing an abnormality on electronic media;
associating the 3-D ultrasound image volume with a body representation;
receiving an operator scan pattern from a MEMS gyro associated with a mock transducer;
tracking position/orientation of the mock transducer in a preselected number of degrees of freedom;
recording the operator scan pattern using the position/orientation;
displaying a 2-D ultrasound image slice from the 3-D composite ultrasound image volume based upon the position/orientation;
receiving an identification of a region of interest associated with the body representation;
assessing if the identification is correct;
recording an amount of time for the identification;
assessing the operator scan pattern by comparing the operator scan pattern with an expert scan pattern; and
providing interactive means for facilitating ultrasound scanning training.

22. The method as in claim 21 further comprising the steps of:
downloading lessons in image-compressed format;
downloading the 3-D ultrasound image volume in image compressed format through a network from a central library; and
storing the lessons and the 3D composite ultrasound image volume on a computer-readable medium in a local library.

23. The method of claim 22 further comprising the steps of:
modifying a display of the 3-D ultrasound image volume corresponding to interactive controls.

24. The method of claim 23 further comprising the steps of:
displaying the location of an image plane in the 3-D ultrasound image volume on a navigational display; and
displaying the scan path based on a digitized representation of the body representation.
